(19) United States
(12) Patent Application Publication
(10) Pub. No.: US 2023/0290522 A1
(43) Pub. Date: Sep. 14, 2023

(54) INFORMATION PROCESSING APPARATUS, NON-TRANSITORY COMPUTER READABLE MEDIUM STORING PROGRAM, AND INFORMATION PROCESSING METHOD

(71) Applicant: FUJIFILM Business Innovation Corp., Tokyo (JP)

(72) Inventors: Ryo MATSUMOTO, Kanagawa (JP); Yusuke TAGUCHI, Kanagawa (JP); Satoshi MARUYAMA, Kanagawa (JP); Masaki WATANABE, Kanagawa (JP); Ayumi TSUJI, Kanagawa (JP)

(73) Assignee: FUJIFILM Business Innovation Corp., Tokyo (JP)

(21) Appl. No.: 17/885,524

(22) Filed: Aug. 10, 2022

(30) Foreign Application Priority Data
Mar. 8, 2022 (JP) 2022-035160

(51) Int. Cl. G16H 80/00; G16H 50/30; G16H 20/10

(52) U.S. Cl. CPC G16H 80/00; G16H 50/30; G16H 20/10

(57) ABSTRACT

An information processing apparatus includes a memory and a processor, in which the memory stores address information representing an address of an institution that has received a prescription, and the processor is configured to, in a case where an abnormality is detected in biological information acquired from a patient, transmit identification information of the patient and information including the fact that the abnormality is detected in the biological information to the address represented by the address information.

### CROSS-REFERENCE TO RELATED APPLICATIONS

This application is based on and claims priority under 35 USC 119 from Japanese Patent Application No. 2022-035160 filed Mar. 8, 2022.

### BACKGROUND

### (i) Technical Field

The present invention relates to an information processing apparatus, a non-transitory computer readable medium storing a program, and an information processing method.

### (ii) Related Art

In JP2014-036747A, a biological information management system is disclosed in which a plurality of pieces of biological information can be acquired efficiently and completely by storing each piece of acquired biological information in association with identification information of a subject as a configuration capable of communicating between a plurality of biological information measurement apparatuses.

In JP2016-091176A, a prescription information transmission system is disclosed in which in a case where prescription information is transmitted to a pharmacy, patient information other than the prescription information is added and transmitted to shorten a waiting time of a patient before medicine delivery.

### SUMMARY

In recent years, a prescription may be sent to a dispensing pharmacy (hereinafter abbreviated as pharmacy) by fax, e-mail, or the like in advance to reduce a waiting time for a patient to receive a medical medicine (hereinafter abbreviated as medicine) at a pharmacy.

However, even though a pharmacist prescribes a wrong medicine at the pharmacy, the patient continues to take the wrongly prescribed medicine, and a patient's medical condition worsens, the pharmacy cannot know that the wrong medicine has been prescribed. In addition, even in a case where a correct medicine is prescribed at the pharmacy, the patient's medical condition may be worsened in a case where the prescribed medicine does not fit patient's constitution.

Aspects of non-limiting embodiments of the present disclosure relate to an information processing apparatus, a non-transitory computer readable medium storing a program, and an information processing method that can inform an institution that prescribes a medicine that an abnormality has occurred in a health condition of a patient who has taken the prescribed medicine.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a memory and a processor in which the memory stores address information representing an address of an institution that has received a prescription and the processor is configured to, in a case where an abnormality is detected in biological information acquired from a patient, transmit identification information of the patient and information including the fact that the abnormality is detected in the biological information to the address represented by the address information.

### BRIEF DESCRIPTION OF THE DRAWINGS

Exemplary embodiment(s) of the present invention will be described in detail based on the following figures, wherein:

FIG. 1 is a diagram showing a system configuration of an information processing system according to an exemplary embodiment of the present invention;

FIG. 2 is a diagram for describing a state where a user who has taken a prescribed medicine at home wears a wearable terminal apparatus 30 on a user's arm and starts the apparatus;

FIG. 3 is a block diagram showing a hardware configuration of a mobile terminal apparatus 10 according to an exemplary embodiment of the present invention;

FIG. 4 is a block diagram showing a functional configuration of the mobile terminal apparatus 10 according to an exemplary embodiment of the present invention;

FIG. 5 is a flowchart for describing an operation of the mobile terminal apparatus 10 according to an exemplary embodiment of the present invention;

FIG. 6 is a sequence chart for describing a state where data is transmitted and received between the wearable terminal apparatus 30, the mobile terminal apparatus 10, and an image forming apparatus 20;

FIG. 7 is a diagram showing an example of an image transmitted by fax from the image forming apparatus 20 to a pharmacy; and

FIG. 8 is a diagram showing a system configuration in a case where a prescription is directly transmitted from a hospital to a pharmacy.

### DETAILED DESCRIPTION

Next, exemplary embodiments of the present invention will be described in detail with reference to drawings.

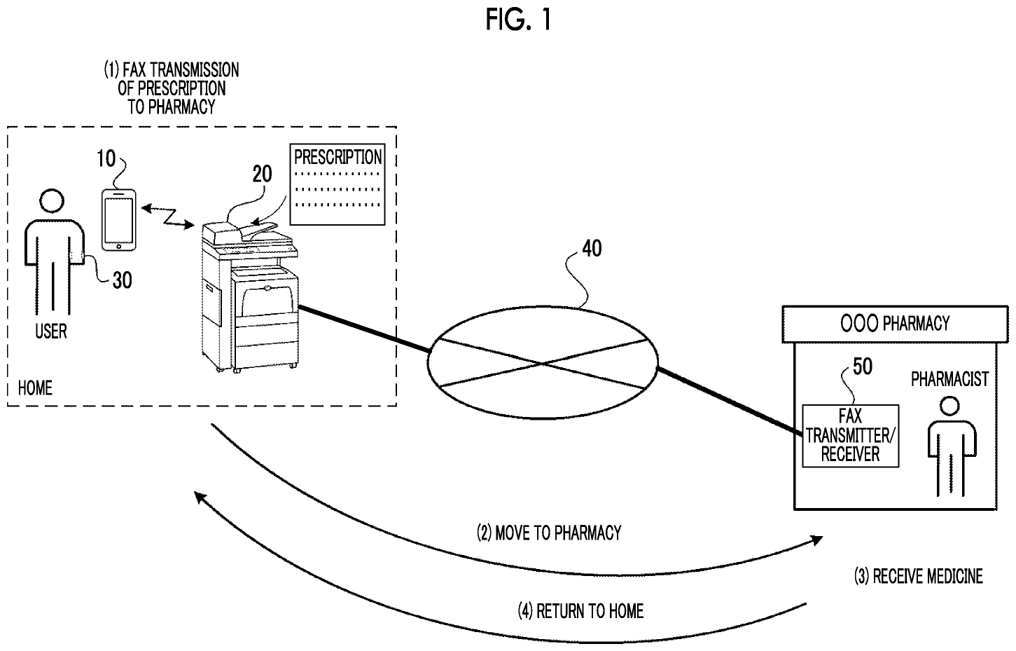

FIG. 1 is a diagram showing a system configuration of an information processing system according to an exemplary embodiment of the present invention.

As shown in FIG. 1, the information processing system according to the exemplary embodiment of the present invention is configured of an image forming apparatus 20 installed at home of a user, a mobile terminal apparatus 10 carried by the user, a wearable terminal apparatus 30 worn on a user's arm to be used, and a fax transmitter/receiver 50 installed in a pharmacy.

The image forming apparatus 20 is a so-called multifunction apparatus having a plurality of functions such as a print function, a scan function, a copy function, and a facsimile function. Further, the image forming apparatus 20 is connected to a public telephone line network 40 and can perform fax transmission to the fax transmitter/receiver 50 installed in the pharmacy via the public telephone line network 40.

The mobile terminal apparatus 10 is an information processing apparatus carried by the user, such as a smartphone. The mobile terminal apparatus 10 can be used by installing various application programs (hereinafter abbreviated as application). Therefore, with installation of an application that provides a user interface for remotely controlling the image forming apparatus 20 on the mobile terminal apparatus 10, the user can control an operation of the image forming apparatus 20 only by operating the mobile terminal apparatus 10.

The wearable terminal apparatus 30 has a function of acquiring biological information, such as pulse and blood pressure, by being worn on the user's arm. The biological information that can be acquired by the wearable terminal apparatus 30 may include information such as blood oxygen saturation (SPO₂) and body temperature. The biological information means information such as a measured value acquired based on biological activity of the user.

A patient can receive a prescription for a medicine by taking a prescription issued by a doctor to the pharmacy. However, in a case where the pharmacy is crowded, a time required to receive the medicine becomes long.

Thus, the prescription may be sent to the pharmacy in advance by fax, e-mail, or the like to reduce a waiting time for the patient to receive the medicine at the pharmacy.

In the information processing system shown in FIG. 1, first, (1) the user sets the prescription in the image forming apparatus 20 installed at home and starts the application of the mobile terminal apparatus 10 to instruct the image forming apparatus 20 to perform the fax transmission. In this case, the user inputs a facsimile number of the pharmacy into the application of the mobile terminal apparatus 10 to instruct the image forming apparatus 20 to perform the fax transmission. With this instruction, the prescription is received by the fax transmitter/receiver 50 of the pharmacy, and a pharmacist starts preparing the medicine at the pharmacy.

Thereafter, the user (2) moves to the pharmacy to which the prescription has been transmitted by fax and (3) receives the medicine prescribed at the pharmacy. Thereafter, (4) the user returns home. Then, the user takes the prescribed medicine at home.

Here, there is no problem as long as a health condition of the user who is the patient who has taken the prescribed medicine is improved by taking the medicine. However, in a case where the pharmacist prescribes a wrong medicine at the pharmacy, the patient continues to take the wrongly prescribed medicine, and the patient's medical condition worsens, the medicine taking is required to be stopped immediately. In addition, even in a case where a correct medicine is prescribed at the pharmacy, the patient's medical condition may be worsened in a case where the prescribed medicine does not fit patient's constitution.

Thus, in the information processing system of the present exemplary embodiment, the processing as shown below is performed to inform an institution that prescribes the medicine that an abnormality has occurred in a health condition of a patient who has taken the prescribed medicine.

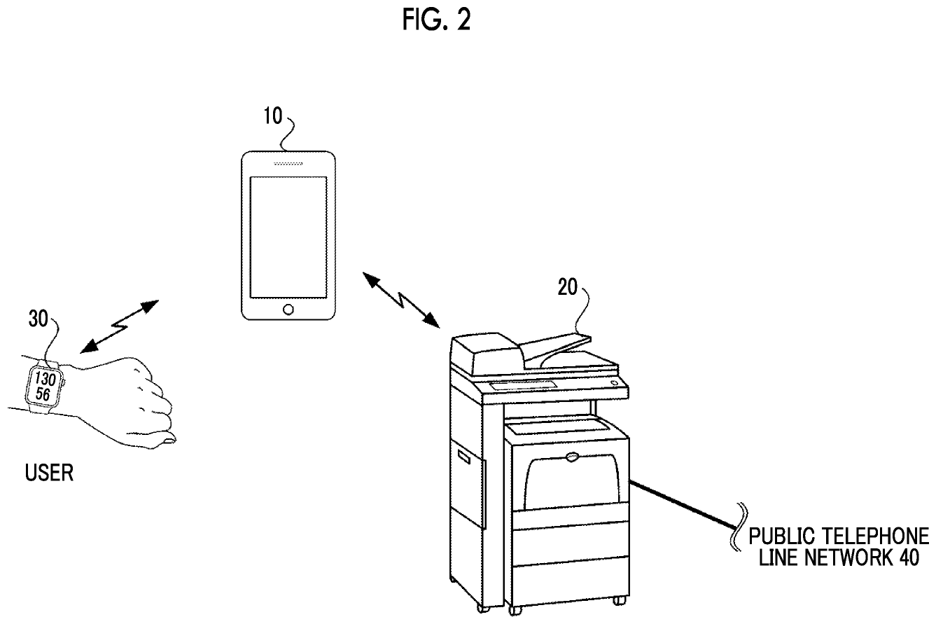

First, the user who has taken the prescribed medicine at home wears the wearable terminal apparatus 30 on the user's arm and starts the apparatus, as shown in FIG. 2. With the start of the apparatus, the wearable terminal apparatus 30 acquires the biological information, such as blood pressure, pulse, and blood oxygen saturation of the user, and monitors whether or not the abnormality occurs in the acquired biological information.

For example, as an example of a case where the abnormality occurs in the acquired biological information, a case will be described in which a systolic blood pressure, which is a highest blood pressure, is 135 mmHg or more or a diastolic blood pressure, which is a lowest blood pressure, is 85 mmHg or more. In this case, the wearable terminal apparatus 30 measures the highest blood pressure and lowest blood pressure of the user and determines that the abnormality has occurred in the health condition of the user in a case where the highest blood pressure is 135 mmHg or more or the lowest blood pressure is 85 mmHg or more.

The wearable terminal apparatus 30 and the mobile terminal apparatus 10 are connected by a wireless communication line such as Bluetooth (registered trademark) or Wi-Fi (registered trademark). Further, the mobile terminal apparatus 10 and the image forming apparatus 20 are also connected by a wireless communication line such as Bluetooth (registered trademark) or Wi-Fi (registered trademark).

The mobile terminal apparatus 10 may be provided with a short-range wireless communication function such as near field communication (NFC). In this case, the mobile terminal apparatus 10 can transmit various instructions to the image forming apparatus 20 via the short-range wireless communication function.

Therefore, in a case where determination is made that the abnormality has occurred in the health condition of the user, the wearable terminal apparatus 30 notifies the mobile terminal apparatus 10 of the occurrence of the abnormality.

In addition to the case where the wearable terminal apparatus 30 acquires the biological information of the user, in a case where another biological information measurement apparatus measures the biological information of the user continuously or periodically and detects the abnormality in the biological information of the user, the notification may be transmitted from the biological information measurement apparatus to the mobile terminal apparatus 10. For example, in a case where a biological information measurement apparatus such as a blood glucose level measurement apparatus, a thermometer, or a pulse oximeter measures the biological information, such as blood glucose level, body temperature, and blood oxygen saturation of the user, and detects the occurrence of the abnormality, the mobile terminal apparatus 10 may be notified.

Further, the biological information measured by the biological information measurement apparatus may be input to the mobile terminal apparatus 10 by the user's operation, and the mobile terminal apparatus 10 may determine whether or not the abnormality occurs in the input biological information.

Since the mobile terminal apparatus 10 issues the transmission instruction to the image forming apparatus 20 to perform the fax transmission of the prescription to the pharmacy, address information representing an address of an institution that has received the prescription is stored as a fax transmission history. Therefore, in a case where the fact that the abnormality has been detected in the biological information acquired from the user who is a patient is received from the wearable terminal apparatus 30, the mobile terminal apparatus 10 instructs the image forming apparatus 20 to perform the fax transmission of identification information, such as a name of the user, and information including the detection of the abnormality in the biological information to the address represented by the address information, that is, the pharmacy that prescribes the medicine.

With this instruction, the image forming apparatus 20 performs the fax transmission of an image including the identification information, such as a name of the user, and the information including the detection of the abnormality in the biological information to the pharmacy via the public telephone line network 40, based on the fax transmission instruction from the mobile terminal apparatus 10. Specifically, the image forming apparatus 20 performs the fax transmission, to the pharmacy, of an image of the prescription that has been transmitted by fax in the past together with a patient's name and contact information, the fact that the abnormality has occurred in a patient's blood pressure value, and the blood pressure value information.

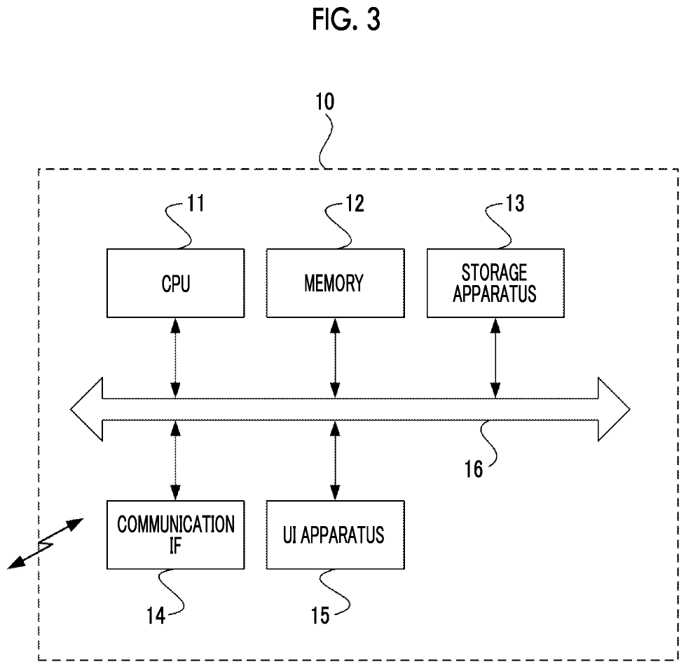

Next, FIG. 3 shows a hardware configuration of the mobile terminal apparatus 10 in the information processing system of the present exemplary embodiment.

As shown in FIG. 3, the mobile terminal apparatus 10 has a CPU 11, a memory 12, a storage apparatus 13 such as a solid state drive (SSD), a communication interface (abbreviated as IF) 14 that transmits and receives data to and from an external apparatus and the like via the wireless communication line, and a user interface (abbreviated as UI) apparatus 15 such as a touch panel or a liquid crystal display. These components are connected to each other via a control bus 16.

The CPU 11 is a processor that executes predetermined processing based on a control program stored in the memory 12 or the storage apparatus 13 to control an operation of the mobile terminal apparatus 10. In the present exemplary embodiment, the CPU 11 reads out and executes the control program stored in the memory 12 or the storage apparatus 13, but the present invention is not limited thereto. This control program may be provided in a form of being recorded on a computer-readable recording medium. For example, this program may be provided in a form of being recorded on optical disks such as a compact disc (CD)-ROM and a digital versatile disc (DVD)-ROM or in a form of being recorded on semiconductor memories such as a universal serial bus (USB) memory and a memory card. Further, this control program may be acquired from an external apparatus via a communication line connected to the communication interface 14.

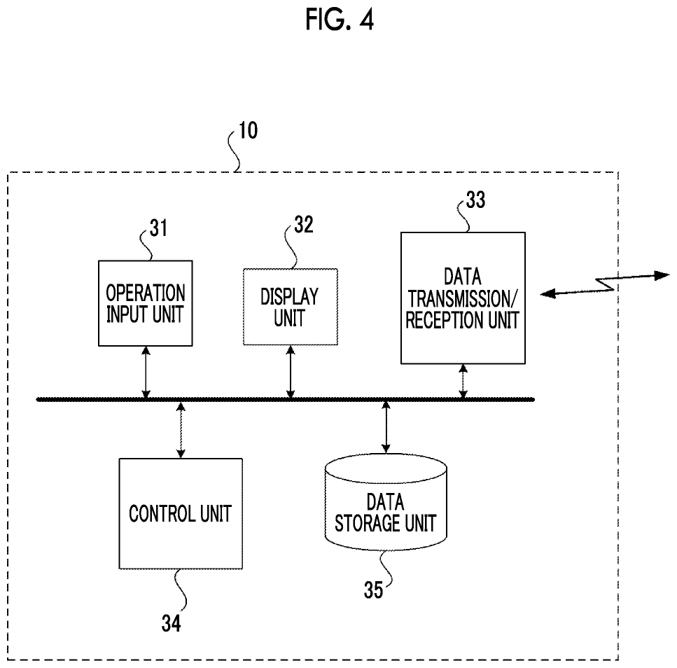

FIG. 4 is a block diagram showing a functional configuration of the mobile terminal apparatus 10 realized by executing the above control program.

As shown in FIG. 4, the mobile terminal apparatus 10 of the present exemplary embodiment comprises an operation input unit 31, a display unit 32, a data transmission/reception unit 33, a control unit 34, and a data storage unit 35.

In the data transmission/reception unit 33, the data is transmitted and received between the wearable terminal apparatus 30 and the image forming apparatus 20 via the wireless communication line. The display unit 32 is controlled by the control unit 34 and displays various pieces of information to the user. In the operation input unit 31, various pieces of operation information performed by the user are input. The control unit 34 causes the data storage unit 35 to store various pieces of data necessary for controlling the operation of the mobile terminal apparatus 10 and controls various operations of the mobile terminal apparatus 10 using the data stored in the data storage unit 35.

The control unit 34 transmits various instructions to the image forming apparatus 20 via the data transmission/reception unit 33 to control the operation of the image forming apparatus 20. Further, the control unit 34 transmits and receives the data to and from the wearable terminal apparatus 30 via the data transmission/reception unit 33 and receives, in a case where the abnormality has occurred in the biological information of the user who is a patient, the notification of the occurrence of the abnormality from the wearable terminal apparatus 30.

The data storage unit 35 stores the address information representing the address of the institution that has received the prescription as fax history information. In a case where the fact that the abnormality is detected in the biological information acquired from the user who is a patient is received from the wearable terminal apparatus 30, the control unit 34 transmits the identification information of the user and the information including the detection of the abnormality in the biological information to the address represented by the address information stored in the data storage unit 35, that is, the pharmacy to which the prescription has been transmitted by fax in the past.

In a case where the abnormality is detected in the biological information acquired from the user, the control unit 34 may include the biological information acquired from the user in the information transmitted to the pharmacy to which the prescription has been transmitted by fax in the past.

Further, in a case where the abnormality is detected in the biological information acquired from the user, the control unit 34 may include information on a prescription content of the prescription in the information transmitted to the pharmacy to which the prescription has been transmitted by fax in the past.

Specifically, in a case where the prescription has been transmitted to the address information designated as image data in past transmission history information, the control unit 34 uses the image data as the information on the prescription content of the prescription to transmit the prescription to the pharmacy to which the prescription has been transmitted by fax in the past.

In a case where the prescription is transmitted from the image forming apparatus 20 to the pharmacy by e-mail or the like, the control unit 34 refers to the transmission history information of the e-mail to transmit, to the pharmacy to which the prescription has been transmitted, the identification information of the user, the fact that the abnormality is detected in the biological information, a measured biological information value, and the information on the prescription content of the prescription.

In a case where the abnormality is detected in the biological information acquired from the user, the control unit 34 refers to the past transmission history information to transmit the identification information of the user and the information including the fact that the abnormality is detected in the biological information to an address with the latest transmission date and time among addresses to which the prescription has been transmitted in the past.

Next, an operation of the information processing system of the present exemplary embodiment will be described with reference to drawings.

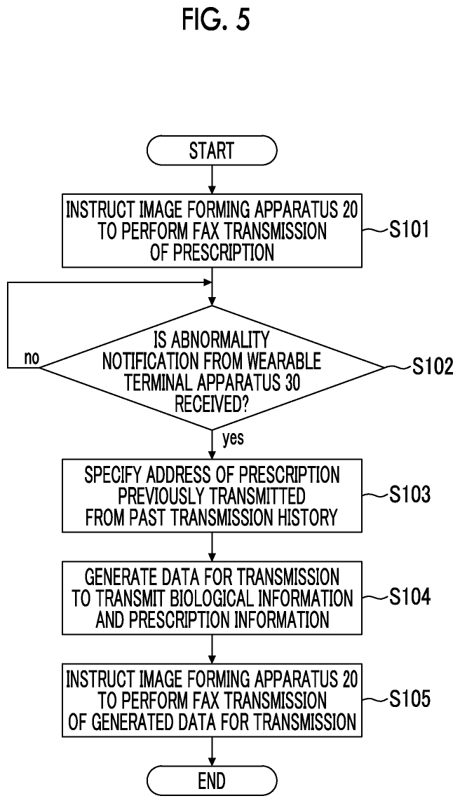

First, the operation of the mobile terminal apparatus 10 will be described with reference to a flowchart in FIG. 5.

First, the user who received the prescription from a hospital returns to user's home, sets the prescription in the image forming apparatus 20, operates the mobile terminal apparatus 10 to input the facsimile number which is the address of the pharmacy, and executes the fax transmission. With the execution, in the mobile terminal apparatus 10, the control unit 34 instructs the image forming apparatus 20 to perform the fax transmission of the prescription to the pharmacy in step S101.

In a case where the prescription is transmitted by fax to the pharmacy, the medicine is dispensed at the pharmacy based on the received prescription. Thereafter, the user goes to this pharmacy to receive the prescribed medicine, returns to user's home, starts the wearable terminal apparatus 30, and wears the apparatus on the user's arm. Then, the wearable terminal apparatus 30 acquires the biological information of the user and starts monitoring whether or not an abnormality occurs in the acquired biological information.

In step S102, the mobile terminal apparatus 10 determines whether or not the abnormality notification from the wearable terminal apparatus 30 is received. In a case where the abnormality notification from the wearable terminal apparatus 30 is not received, the control unit 34 repeats this processing. In a case where the abnormality notification from the wearable terminal apparatus 30 is received in step S102, the control unit 34 specifies the address of the prescription previously transmitted from the past transmission history, specifically, the fax transmission history in step S103.

In step S104, the control unit 34 generates data for transmission to transmit the biological information from the wearable terminal apparatus 30, the fact that the abnormality has occurred in the measured biological information, the transmitted prescription information, and the user information such as the name and contact information of the user.

In step S105, the control unit 34 instructs the image forming apparatus 20 to perform the fax transmission of the data for transmission generated in step S104 to the address specified in step S103.

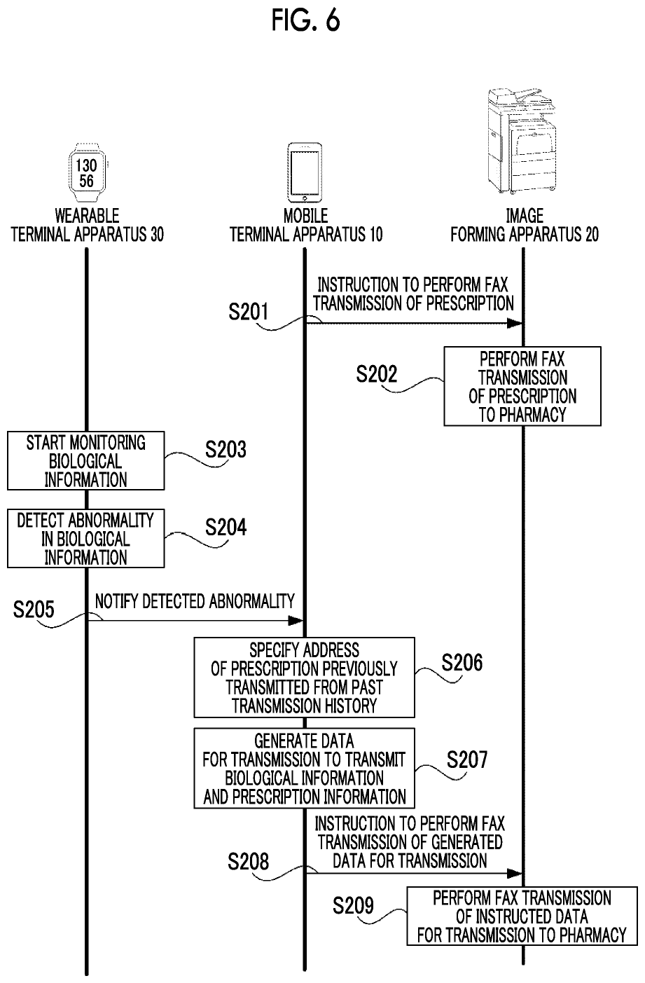

Next, a state where the data is transmitted and received between the wearable terminal apparatus 30, the mobile terminal apparatus 10, and the image forming apparatus 20 will be described with reference to a sequence chart in FIG. 6.

First, in step S201, the mobile terminal apparatus 10 instructs the image forming apparatus 20 to perform the fax transmission of the prescription. With the instruction, in step S202, the image forming apparatus 20 performs the fax transmission of the prescription to the pharmacy.

Thereafter, in a case where the user who has received and taken the medicine prescribed by the pharmacy wears the wearable terminal apparatus 30, the wearable terminal apparatus 30 starts monitoring the biological information of the user in step S203. In a case where the abnormality is detected in the acquired biological information in step S204, the wearable terminal apparatus 30 notifies the mobile terminal apparatus 10 of the detected abnormality in step S205.

With the notification, in step S206, the mobile terminal apparatus 10 specifies the address of the prescription previously transmitted from the past transmission history, specifically, the fax transmission history.

In step S207, the mobile terminal apparatus 10 generates the data for transmission to transmit the biological information from the wearable terminal apparatus 30, the fact that the abnormality has occurred in the measured biological information, the transmitted prescription information, and the user information such as the name and contact information of the user.

Thereafter, in step S208, the mobile terminal apparatus 10 instructs the image forming apparatus 20 to perform the fax transmission of the generated transmission data to the address specified in step S206.

The image forming apparatus 20 that has received the fax transmission instruction from the mobile terminal apparatus 10 performs the fax transmission of the instructed data for transmission to the pharmacy in step S209.

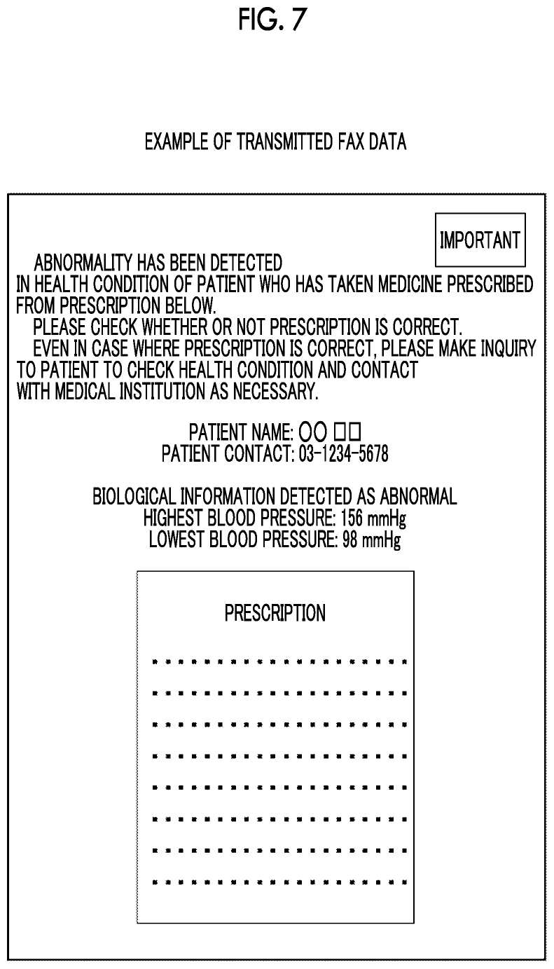

FIG. 7 shows an example of an image transmitted by fax from the image forming apparatus 20 to the pharmacy in this manner.

FIG. 7 shows an image including information such as the fact that the abnormality has been detected in the health condition of the patient who has taken the medicine and the fact that a recheck of the prescription content of the prescription is requested, the patient's name and contact information, and the biological information values such as the blood pressure values detected as abnormal, together with the image of the prescription transmitted to the pharmacy in the past. The pharmacy that has received the fax as shown in FIG. 7 examines the past prescription history and investigates whether or not the prescribed medicine is correct. In a case where the prescribed medicine is incorrect, the pharmacy can immediately contact the user to cause the user to stop taking the medicine.

In addition, even in a case where the medicine prescribed at the pharmacy is correct, the medicine may not fit the user's constitution and the health condition may worse. Thus, an inquiry is made to the user from the pharmacy to interrupt the medicine taking, contact with the doctor, or the like as necessary.

Case where Prescription Is Directly Transmitted from Hospital to Pharmacy

In the exemplary embodiment described above, the prescription issued by the doctor is transmitted in advance to the pharmacy by the user who is a patient. However, a system may be possible in which the prescription is directly transmitted from the hospital to the pharmacy.

In this case, a system configuration may be possible in which the patient receives a medical examination by the doctor online from home, as in telemedicine, instead of a case where the patient goes to the hospital to receive the medical examination by the doctor directly, and the prescription is transmitted from the hospital to the pharmacy based on a doctor's instruction.

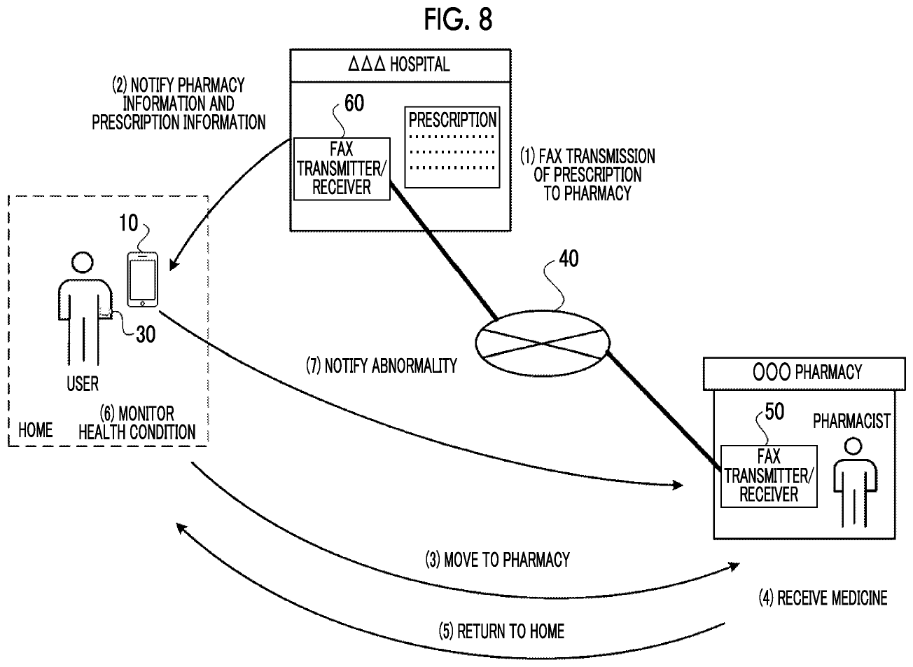

FIG. 8 shows a system configuration in a case where the prescription is directly transmitted from the hospital to the pharmacy in this manner.

In the system configuration shown in FIG. 8, (1) first, the prescription is transmitted by fax from the fax transmitter/receiver 60 of the hospital to the fax transmitter/receiver 50 of the pharmacy via the public telephone line network 40. (2) Then, the hospital system notifies the mobile terminal apparatus 10 of the patient which is the user of information such as an address and name of the pharmacy to which the prescription has been transmitted by fax and information such as the prescription content of the prescription.

Thereafter, the user (3) moves to the pharmacy to which the prescription has been transmitted by fax and (4) receives the medicine prescribed at the pharmacy. Thereafter, (5) the user returns home. Then, the user takes the prescribed medicine at home.

The user who has taken the prescribed medicine at home wears the wearable terminal apparatus 30 on the user's arm and starts the apparatus. (6) Then, the wearable terminal apparatus 30 acquires the biological information such as blood pressure, pulse, and blood oxygen saturation of the user and monitors whether or not the abnormality occurs in the acquired biological information.

Thereafter, in a case where the wearable terminal apparatus 30 detects the abnormality in the biological information of the user, the wearable terminal apparatus 30 notifies the mobile terminal apparatus 10 that the abnormality has been detected.

(7) As a result, the mobile terminal apparatus 10 notifies the pharmacy that the abnormality has occurred in the health condition of the user. In this case, the mobile terminal apparatus 10 may notify the pharmacy by e-mail or may notify the pharmacy by fax transmission.

As described above, in the mobile terminal apparatus 10 of the present exemplary embodiment, the control unit 34 acquires, from the hospital system, the information on the prescription content of the transmitted prescription together with the address information representing a destination of the pharmacy which is the institution that has received the prescription and stores the acquired information in the data storage unit 35. In a case where the abnormality has been detected in the biological information acquired from the user, the control unit 34 transmits information including the information on the prescription content of the transmitted prescription to the address represented by the address information stored in the data storage unit 35.

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

### Modification Example

In the exemplary embodiment described above, the case where the present invention is applied to the mobile terminal apparatus such as the smartphone has been described, but the present invention is not limited thereto. The present invention can be similarly applied even in a case where other information processing apparatuses such as a personal computer and a tablet terminal apparatus are used.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing apparatus comprising:
a memory; and
a processor,
wherein the memory stores address information representing an address of an institution that has received a prescription, and
the processor is configured to:
in a case where an abnormality is detected in biological information acquired from a patient, transmit identification information of the patient and information including the fact that the abnormality is detected in the biological information to the address represented by the address information.

2. The information processing apparatus according to claim 1, wherein the processor is configured to:
in a case where an abnormality is detected in biological information acquired from a patient, include the biological information acquired from the patient in the information to be transmitted to the address represented by the address information.

3. The information processing apparatus according to claim 1, wherein the processor is configured to:
in a case where an abnormality is detected in biological information acquired from a patient, include information on a prescription content of the prescription in the information to be transmitted to the address represented by the address information.

4. The information processing apparatus according to claim 2, wherein the processor is configured to:
in a case where an abnormality is detected in biological information acquired from a patient, include information on a prescription content of the prescription in the information to be transmitted to the address represented by the address information.

5. The information processing apparatus according to claim 3, wherein the processor is configured to:
in a case where a prescription has been transmitted to designated address information as image data in past transmission history information, include the image data in the information to be transmitted to the address represented by the address information as the information on the prescription content of the prescription.

6. The information processing apparatus according to claim 4, wherein the processor is configured to:
in a case where a prescription has been transmitted to designated address information as image data in past transmission history information, include the image data in the information to be transmitted to the address represented by the address information as the information on the prescription content of the prescription.

7. The information processing apparatus according to claim 1, wherein the processor is configured to:
in a case where an abnormality is detected in biological information acquired from a patient, refer to past transmission history information to transmit the identification information of the patient and the information including the fact that the abnormality is detected in the biological information to an address with a latest transmission date and time among addresses to which a prescription has been transmitted in the past.

8. The information processing apparatus according to claim 1, wherein the processor is configured to:
acquire address information representing an address of an institution that has received a prescription from another system, and
store the address information in the memory.

9. The information processing apparatus according to claim 8, wherein the processor is configured to:
acquire information on a prescription content of the transmitted prescription from the other system together with the address information representing the address of the institution that has received the prescription,
store the acquired information in the memory, and
in a case where an abnormality is detected in biological information acquired from a patient, transmit information including the information on the prescription content of the transmitted prescription to the address represented by the address information.

10. A non-transitory computer readable medium storing a program causing a computer to execute a process comprising:
storing address information representing an address of an institution that has received a prescription; and
transmitting, in a case where an abnormality is detected in biological information acquired from a patient, identification information of the patient and information including the fact that the abnormality is detected in the biological information to the address represented by the address information.

11. An Information processing method comprising:
storing address information representing an address of an institution that has received a prescription; and
transmitting, in a case where an abnormality is detected in biological information acquired from a patient, identification information of the patient and information including the fact that the abnormality is detected in the biological information to the address represented by the address information.
